SEQUENCE LISTING (9 sequences)

SEQ ID NO: 1 - DNA, 40 bp, Homo sapiens
cagatgagaacaaggaaaatctttcctcaaatgaaagaga

SEQ ID NO: 2 - DNA, 2299 bp, Homo sapiens
  CDS (460)..(1911)
cgaaaggccggccttggctgcgacagcctgggtaagaggtgtaggtcggcttggttttctgctacccggagctgggcaagcgggttgggagaacagcgaagacagcgtgagcctgggccgttgcctcgaggctctcgcccggcttctcttgccgacccgccacgtttgtttggatttaatcttacagctggttgccggcgcccgcccgcccgctggcctcgcggtgtgagagggaagcacccgtgcctgtggctggtggctggcgcctggagggtccgcacacccgcccggccgcgccgctttgcccgcggcagccgcgtccctgaaccgcggagtcgtgtttgtgtttgacccgcgggcgccggtggcgcgcggccgaggccggtgtcggcggggcggggcggtcgcggcggaggcagaggaagagggagcgggagctctgcgaggccgggcgccgccatggaactgggcccgagccccgcaccgcgccgcctgctcttcgcctgcagcccccctcccgcgtcgcagcccgtcgtgaaggcgctatttggcgcttcagccgccgggggactgtcgcctgtcaccaacctgaccgtcactatggaccagctgcagggtctgggcagtgattatgagcaaccactggaggtgaagaacaacagtaatctgcagagaatgggctcctccgagtcaacagattcaggtttctgtctagattctcctgggccattggacagtaaagaaaaccttgaaaatcctatgagaagaatacattccctacctcaaaagctgttgggatgtagtccagctctgaagaggagccattctgattctcttgaccatgacatctttcagctcatcgacccagatgagaacaaggaaaatctttcctcaaatgaaagagatagcagtgaaccagggaatttcattcctctttttacaccccagtcacctgtgacagccactttgtctgatgaggatgatggcttcgtggaccttctcgatggagagaatctgaagaatgaggaggagaccccctcgtgcatggcaagcctctggacagctcctctcgtcatgagaactacaaaccttgacaaccgatgcaagctgtttgactccccttccctgtgtagctccagcactcggtcagtgttgaagagaccagaacgttctcaagaggagtctccacctggaagtacaaagaggaggaagagcatgtctggggccagccccaaagagtcaactaatccagagaaggcccatgagactcttcatcagtctttatccctggcatcttcccccaaaggaaccattgagaacattttggacaatgacccaagggaccttataggagacttctccaagggttatctctttcatacagttgctgggaaacatcaggatttaaaatacatctctccagaaattatggcatctgttttgaatggcaagtttgccaacctcattaaagagtttgttatcatcgactgtcgatacccatatgaatacgagggaggccacatcaagggtgcagtgaacttgcacatggaagaagaggttgaagacttcttattgaagaagcccattgtacctactgatggcaagcgtgtcattgttgtgtttcactgcgagttttcttctgagagaggtccccgcatgtgccggtatgtgagagagagagatcgcctgggtaatgaataccccaaactccactaccctgagctgtatgtcctgaaggggggatacaaggagttctttatgaaatgccagtcttactgtgagccccctagctaccggcccatgcaccacgaggactttaaagaagacctgaagaagttccgcaccaagagccggacctgggcaggggagaagagcaagagggagatgtacagtcgtctgaagaagctctgagggcggcaggaccagccagcagcagcccaagcttccctccatccccctttaccctctttcctgcagagaaacttaagcaaaggggacagctgtgtgacatttggagagggggcctgggacttccatgccttaaacctacctcccacactcccaaggttggagcccagggcatcttgctggctacgcctcttctgtccctgttagacgtcctccgtccatatcagaactgtgccacaatgcagttctgagcaccgtgtcaagctgctctgagccacagtgggatgaaccagccggggccttatcgggctccagcatctcatgaggggagaggagacggaggggagtagagaagtttacacagaaatgctgctggccaaatagcaaagag

SEQ ID NO: 3 - protein, 483 aa, Homo sapiens
MELGPSPAPRRLLFACSPPPASQPVVKALFGASAAGGLSPVTNLTVTMDQLQGLGSDYEQPLEVKNNSNLQRMGSSESTDSGFCLDSPGPLDSKENLENPMRRIHSLPQKLLGCSPALKRSHSDSLDHDIFQLIDPDENKENLSSNERDSSEPGNFIPLFTPQSPVTATLSDEDDGFVDLLDGENLKNEEETPSCMASLWTAPLVMRTTNLDNRCKLFDSPSLCSSSTRSVLKRPERSQEESPPGSTKRRKSMSGASPKESTNPEKAHETLHQSLSLASSPKGTIENILDNDPRDLIGDFSKGYLFHTVAGKHQDLKYISPEIMASVLNGKFANLIKEFVIIDCRYPYEYEGGHIKGAVNLHMEEEVEDFLLKKPIVPTDGKRVIVVFHCEFSSERGPRMCRYVRERDRLGNEYPKLHYPELYVLKGGYKEFFMKCQSYCEPPSYRPMHHEDFKEDLKKFRTKSRTWAGEKSKREMYSRLKKL

SEQ ID NO: 4 - DNA, 20 bp, Homo sapiens
caaggaaaatctttcctcaa

SEQ ID NO: 5 - protein, 10 aa, Homo sapiens
ENKENLSSNE

SEQ ID NO: 6 - DNA, 28 bp, Homo sapiens
ctcatcgacccagatgagaacaaggaaa

SEQ ID NO: 7 - DNA, 28 bp, Homo sapiens
tcctgatgtttcccagcaactgtatgaa

SEQ ID NO: 8 - DNA, 28 bp, Homo sapiens
atggaactgggcccgagccccgcaccgc

SEQ ID NO: 9 - DNA, 28 bp, Homo sapiens
tcagagcttcttcagacgactgtacatc

(19) United States
(12) Patent Application Publication
(10) Pub. No.: US 2005/0176125 A1
(43) Pub. Date: Aug. 11, 2005

(54) Alternatively spliced isoform of CDC25A phosphatase and methods of use

(75) Inventors: John Castle, Seattle, WA (US); Christopher Armour, Kirkland, WA (US); Philip W. Garrett-Engele, Seattle, WA (US); Zhengyan Kan, Bellevue, WA (US); Christopher K. Raymond, Seattle, WA (US); Nicholas F. Tsinoremas, West Palm Beach, FL (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Rosetta Inpharmatics LLC, Seattle, WA (US)

(21) Appl. No.: 11/022,042

(22) Filed: Dec. 22, 2004

Related U.S. Application Data
(60) Provisional application No. 60/531,860, filed on Dec. 23, 2003.

(51) Int. Cl. C07H 21/04; C12N 9/16

(52) U.S. Cl. 435/196; 536/23.2

(57) ABSTRACT

The present invention features nucleic acids and polypeptides encoding a novel splice variant isoform of cell division cycle 25A (Cdc25A) phosphatase. The polynucleotide sequence of Cdc25Asv1 is provided by SEQ ID NO:2. The amino acid sequence for Cdc25Asv1 is provided by SEQ ID NO:3. The present invention also provides methods for using Cdc25Asv1 polynucleotides and proteins to screen for compounds that bind to Cdc25Asv1.

### CROSS-REFERENCE(S) TO RELATED APPLICATION

This application claims the benefit of U.S. Provisional Application No. 60/531,860 filed Dec. 23, 2003.

### FIELD OF THE INVENTION

The present invention relates to a novel isoform of a human protein, and particularly relates to a novel isoform of the Cdc25A phosphatase and methods of use.

### BACKGROUND OF THE INVENTION

The references cited herein are not admitted to be prior art to the claimed invention.

The entrance of eukaryotic cells into mitosis from G2 is a highly regulated event that is initiated following the activation of a protein kinase known as the M-phase kinase (MPF) (see Hunt, Curr. Opinion Cell. Biol. 1:268-274, 1989). MPF consists of at least three subunits, including a catalytic subunit (cell division cycle 2 (“Cdc2”) kinase), a regulatory subunit (Cyclin B), and a low molecular weight subunit (p13-Suc1) (Brizuela, L., et al., EMBO J. 6:3507-3514, 1987). Once activated, MPF triggers a cascade of downstream mitotic events by directly phosphorylating a wide variety of mitotic substrates such as nuclear lamins, histones, and microtubule-associated proteins (see, e.g., Moreno et al., Cell 61:549-551, 1990).

The MPF enzyme complex is subject to several levels of regulation. One mechanism of regulation involves the inhibitory phosphorylation of Cdc2 on Tyr-15 and Thr-14, two residues located in the ATP binding site of the kinase (Draetta, G., et al., Nature 336:738-744, 1988). A stimulatory phosphatase, known as cell division cycle 25 (“Cdc25”), is responsible for Cdc2 dephosphorylation at Tyr-15 and Thr-14 and serves as a rate-limiting mitotic activator in eukaryotic cells (Gauteir, J. et al., Cell 67:197-211, 1991). In humans, Cdc25 is a multigene family, including Cdc25A, Cdc25B and Cdc25C. The three human Cdc25 proteins share approximately 40% identity in the most conserved C-terminal region (see U.S. Pat. No. 5,441,880). Cdc25A and Cdc25B each exhibit endogenous tyrosine phosphatase activity that can be specifically activated by B-type cyclin (see U.S. Pat. No. 5,441,880).

To protect genome integrity, eukaryotic cells exposed to genotoxic stress cease proliferating to provide time for DNA repair. At least two states in the cell cycle are regulated in response to DNA damage; the G1/S and the G2/M transitions. In vivo experiments have shown that Cdc25A protein is degraded in response to activation of the DNA damage checkpoint (Mailand et al., Science 288:1425-1429, 2000). The inhibition of Cdc25A in response to DNA damage is dependent on the checkpoint 1 (“Chk1”) kinase which phosphorylates Cdc25A (Furnari et al., Science 277:1495-1497, 1997). Studies have also shown that activation of the DNA damage checkpoint results in reduced nuclear localization of Cdc25A (Lopez-Girona et al., Nature 397:172-175, 1999). This effect on localization is blocked by mutation of Chk1 phosphoylation sites on Cdc25A, suggesting the effect is dependent on Chkl phosphorylation (Zeng et al., Mol. Cell. Biol. 19:7410-7419, 1999).

Given the role the Cdc25 phosphatases have in promoting cell division, the genes encoding these phosphatases are implicated as potential oncogenes. In particular, human Cdc25A is implicated as a drug target for cancer therapy because overexpression of Cdc25A bypasses the cell cycle arrest induced by DNA damage, leading to enhanced DNA damage and decreased cell survival (Mailand et al., 2000). The human Cdc25A has been mapped to 3p21, an area which is frequently involved in karyotypic abnormalities in renal carcinomas, small cell carcinomas of the lung and benign tumors of the salivary gland (Demetrick et al., Genomics 18:144-147 (1993)). Moreover, overexpression of Cdc25A phosphatase has been observed in head and neck cancers (Gasparotto et al., Cancer Res. 57:2366-2368 (1997), non-small cell lung cancer (Wu et al., Cancer Res. 58:4082-4085 (1998), and correlates with poor prognosis in hepatocellular carcinoma (Xu et al., Clin. Cancer Res. 9:1764-1772 (2003). Because of the multiple therapeutic values of drugs targeting the regulators and checkpoints of the eukaryotic cell cycle, and the essential role played by Cdc25A, there is a need in the art to identify new isoform variants of Cdc25A and for compounds that selectively bind to isoforms of Cdc25A. The present invention is directed towards a novel Cdc25A isoform (Cdc25Asv1) and uses thereof.

### SUMMARY OF THE INVENTION

Microarray experiments and RT-PCR have been used to identify and confirm the presence of a novel splice variant of human Cdc25A mRNA. More specifically, the present invention features polynucleotides encoding Cdc25Asv1, a protein isoform of Cdc25A. A novel polynucleotide junction resulting from the splicing of exon 5 to exon 7 is provided by SEQ ID NO:1. A polynucleotide sequence encoding Cdc25Asv1 is provided by SEQ ID NO:2. An amino acid sequence for Cdc25Asv1 is provided by SEQ ID NO:3.

Thus, a first aspect of the present invention describes purified Cdc25Asv1 encoding nucleic acid molecules. The Cdc25Asv1 encoding nucleic acid molecules comprise SEQ ID NO:2 or the complement thereof. Reference to the presence of one region does not indicate that another region is not present. For example, in different embodiments the inventive nucleic acid molecule can comprise, consist, or consist essentially of a nucleic acid sequence encoding SEQ ID NO:3.

Another aspect of the present invention describes a purified Cdc25Asv1 polypeptide that can comprise, consist, or consist essentially of the amino acid sequence of SEQ ID NO:3.

Another aspect of the present invention describes expression vectors. In one embodiment of the present invention, the inventive expression vector comprises a nucleotide sequence encoding a polypeptide comprising, consisting, or consisting essentially of SEQ ID NO:3, wherein the nucleotide sequence is transcriptionally coupled to an exogenous promoter. In another embodiment, the nucleotide sequence comprises, consists of, or consists essentially of SEQ ID NO:2 and is transcriptionally coupled to an exogenous promoter.

Another aspect of the present invention describes recombinant cells comprising expression vectors comprising, consisting of, or consisting essentially of the above-described sequences and the promoter is recognized by an RNA polymerase present in the cell.

Another aspect of the present invention describes a recombinant cell made by a process comprising the step of introducing into the cell an expression vector comprising a nucleotide sequence comprising, consisting, or consisting essentially of SEQ ID NO:2, or a nucleotide sequence encoding a polypeptide comprising, consisting of, or consisting essentially of an amino acid sequence of SEQ ID NO:3, wherein the nucleotide sequence is transcriptionally coupled to an exogenous promoter. The expression vector can be used to insert recombinant nucleic acid into the host genome or can exist as an autonomous nucleic acid molecule.

Another aspect of the present invention provides a method of producing Cdc25Asv1 polypeptide comprising SEQ ID NO:3. The method involves the step of growing a recombinant cell containing an inventive expression vector under conditions wherein the polypeptide is expressed from the expression vector.

Another aspect of the present invention features a purified antibody preparation comprising an antibody that binds selectively to Cdc25Asv1 as compared to one or more Cdc25A isoform polypeptides that are not Cdc25Asv1.

Another aspect of the present invention provides a method of screening for a compound that binds to Cdc25Asv1, or fragments thereof. In one embodiment, the method comprises the steps of: (a) expressing a polypeptide comprising the amino acid sequence of SEQ ID NO:3 or a fragment thereof from recombinant nucleic acid; (b) providing to said polypeptide a labeled Cdc25A ligand that binds to said polypeptide and a test preparation comprising one or more test compounds; (c) and measuring the effect of said test preparation on binding of said test preparation to said polypeptide comprising SEQ ID NO:3. In another embodiment of the method, a compound is identified that binds selectively to Cdc25Asv1 polypeptide as compared to one or more Cdc25A isoform polypeptides that are not Cdc25Asv1. This method comprises the steps of: providing a Cdc25Asv1 polypeptide comprising SEQ ID NO:3; providing a Cdc25A isoform polypeptide that is not Cdc25Asv1; contacting said Cdc25Asv1 polypeptide and said Cdc25A isoform polypeptide that is not Cdc25Asv1 with a test preparation comprising one or more test compounds; and determining the binding of said test preparation to said Cdc25Asv1 polypeptide and to Cdc25A isoform polypeptide that is not Cdc25Asv1, wherein a test preparation that binds to said Cdc25Asv1 polypeptide but does not bind to said Cdc25A isoform polypeptide that is not Cdc25Asv1 contains a compound that selectively binds said Cdc25A polypeptide.

In another embodiment of the invention a method is provided for screening for a compound able to bind to or interact with a Cdc25Asv1 protein or a fragment thereof comprising the steps of: expressing a Cdc25Asv1 polypeptide comprising SEQ ID NO:3 or a fragment thereof from a recombinant nucleic acid; providing to said polypeptide a labeled Cdc25A ligand that binds to said polypeptide and a test preparation comprising one or more compounds; and measuring the effect of said test preparation on binding of said labeled Cdc25A ligand to said polypeptide, wherein a test preparation that alters the binding of said labeled Cdc25A ligand to said polypeptide contains a compound that binds to or interacts with said polypeptide.

Other features and advantages of the invention will become apparent from the additional descriptions provided herein, including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A illustrates the exon structure of Cdc25A mRNA corresponding to the known long reference form of Cdc25A mRNA (labeled NM_{—}001789.1) and the exon structure corresponding to the inventive short form splice variant (labeled Cdc25Asv1).

FIG. 1B depicts the nucleotide sequences of the exon junctions resulting from the splicing of exon 5 to exon 7 (SEQ ID NO:1) in Cdc25Asv1 mRNA. In FIG. 1B, the nucleotides shown in italics represent the 20 nucleotides at the 3′ end of exon 5 and the nucleotides shown in underline represent the 20 nucleotides at the 5′ end of exon 7.

### DETAILED DESCRIPTION OF THE PREFERRED EMBODIMENT

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

Unless defined otherwise, all technical and scientific terms used herein have the meaning commonly understood by one of ordinary skill in the art to which this invention belongs.

As used herein, “Cdc25A” refers to human cell division cycle 25A protein (NM_{—}001789.1). In contrast, reference to a Cdc25A isoform, includes NM_{—}001789.1 and other polypeptide isoform variants of Cdc25A.

As used herein, “Cdc25Asv1” refers to a splice variant isoform of human Cdc25A protein, wherein the splice variant has the amino acid sequence set forth in SEQ ID NO:3.

As used herein, “Cdc25A” refers to polynucleotides encoding Cdc25A.

As used herein, “Cdc25Asv1” refers to polynucleotides encoding Cdc25Asv1 having an amino acid sequence set forth in SEQ ID NO:3.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or intemucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect to the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in an isolated nucleic acid sample or preparation. Reference to “purified nucleic acid” does not require that the nucleic acid has undergone any purification and may include, for example, chemically synthesized nucleic acid that has not been purified.

The phrases “isolated protein,” “isolated polypeptide,” “isolated peptide,” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature; where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation.

As used herein, a “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN:3540641513)). As used herein, antibodies can be produced by any known technique, including harvest from cell culture of native B-lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense,” as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject,” as used herein refers to an organism and to cells or tissues derived therefrom. For example the organism may be an animal, including but not limited to animals such as cows, pigs, horses, chickens, cats, dogs, etc., and is usually a mammal, and most commonly human.

The present invention relates to the nucleic acid sequences encoding human Cdc25Asv1, which is an alternatively spliced isoform of Cdc25A, and to the amino acid sequences encoding this protein. A novel polynucleotide junction resulting from the splicing of exon 5 to exon 7 is provided by SEQ ID NO:1. SEQ ID NO:2 is a polynucleotide sequence representing an exemplary open reading frame that encodes the Cdc25Asv1 protein. SEQ ID NO:3 shows the polypeptide sequence of Cdc25Asv1.

The Cdc25Asv1 polynucleotide sequence encoding Cdc25Asv1 protein, as exemplified and enabled herein includes a number of specific, substantial and credible utilities. For example, Cdc25Asv1 encoding nucleic acids were identified in a mRNA sample obtained from a human source (see Example 1). Such nucleic acids can be used as hybridization probes to distinguish between cells that produce Cdc25Asv1 transcripts from human or non-human cells (including bacteria) that do not produce such transcripts. Similarly, antibodies specific for Cdc25Asv1 can be used to distinguish between cells that express Cdc25Asv1 from human or non-human cells (including bacteria) that do not express Cdc25Asv1.

Cdc25A is an important drug target for modulating (inhibiting or enhancing) cellular proliferation and for preserving genomic stability after cellular exposure to genotoxic agents (see Mailand et al., Science 288:1425-1429, 2000). Given the pivotal role Cdc25A plays in regulation of cell cycle progression, it is of value to identify Cdc25A isoforms and identify Cdc25A ligand compounds that are isoform specific, as well as compounds that are effective ligands for two or more isoforms. In some embodiments of the present invention, it is important to identify compounds that modulate a specific Cdc25A isoform activity, yet do not bind to or interact with a plurality of different Cdc25A isoforms. Compounds that bind to or interact with multiple Cdc25A isoforns may require higher drug doses to saturate multiple Cdc25A isoform binding sites and thereby result in a greater likelihood of secondary non-therapeutic side effects. Furthermore, biological effects could also be caused by the specific interaction of a drug with the Cdc25Asv1 isoform.

Cdc25A is known to be an important substrate for Chk1 (see Xiao et al., J. Biol. Chem. 278:21767-21773, 2003). Therefore, Cdc25A isoforms, including Cdc25Asv1 can be used to screen potential therapeutic agents directed to Chk1. The presence of particular Cdc25A isoforms can also be used as pharmacodynamic markers to predict efficacy for a particular Chk1 therapeutic.

Because Cdc25A is overexpressed in certain tumor types, (such as, for example, head and neck cancers (Gasparotto et al., Cancer Res. 57:2366-2368 (1997), non-small cell lung cancer (Wu et al., Cancer Res. 58:4082-4085 (1998), and hepatocellular carcinoma (Xu et al., Clin. Cancer Res. 9:1764-1772 (2003)), the analysis of CdC25Asv1 is useful for diagnosing particular tumor types. For example, a gene probe specific for a Cdc25A isoform can be used to detect and quantify the Cdc25A isoform expression levels in tumor cells. For detection of Cdc25A polypeptide isoforms, antibodies specific for the Cdc25A isoform may be used. The presence and expression level of Cdc25A isoforms may also be used to determine prognosis of certain cancer types, such as for example, hepatocellular carcinoma (Xu et al., Clin. Cancer Res. 9:1764-1772 (2003). Cdc25A isoforms are also useful as markers for screening the efficacy of various antimitotic compounds used in cancer therapy, such as, for example, actinomycin D, carboplatin, cis-platinum, etoposide, fluoro-uracil, and methotrexate.

Cdc25A variants may also be used as a tool to further expand the knowledge of the systems biology of cell cycle regulation. In particular, Cdc25A variants can be used to provide a set of pharmacogenomic and proteomic markers to track particular disease states such as cancer in individual subjects.

For the foregoing reasons, the Cdc25Asv1 protein represents a useful compound binding target and has utility in the identification of new Cdc25A ligands exhibiting a preferred specificity profile and having greater efficacy for their intended use.

In some embodiments, Cdc25Asv1 activity is modulated by a ligand compound to achieve anti-proliferative effects such as one or more of the following: preventing or reducing the risk of occurrence, or recurrence of diseases resulting from cellular proliferation, such as cancer and inflammatory diseases. Compounds that treat cancers are particularly important because of the cause-and-effect relationship between cancers and mortality.

Compounds capable of modulating Cdc25Asv1 include agonists, antagonists, and allosteric modulators of Cdc25Asv1. Inhibitors of Cdc25A achieve clinical efficacy by a number of known and unknown mechanisms. While not wishing to be limited to any particular theory of therapeutic efficacy, generally, but not always, Cdc25Asv1 compounds may be used to inhibit Cdc25A phosphatase activity, and thereby inhibit Cdc2 kinase activity, leading to inhibition of cellular proliferation. Therefore, agents that modulate Cdc25A activity may be used to achieve a therapeutic benefit for any disease or condition due to, or exacerbated by, abnormal levels of Cdc25A protein or its activity.

Cdc25Asv1 can also be affected by modulating the cellular abundance of transcripts encoding Cdc25Asv1. Compounds modulating the abundance of transcripts encoding Cdc25Asv1 include a cloned polynucleotide encoding Cdc25Asv1 that is capable of expressing Cdc25Asv1 in vivo, antisense nucleic acids targeted to Cdc25Asv1 transcripts, and enzymatic nucleic acids, such as ribozymes and RNAi, targeted to Cdc25A transcripts.

In some embodiments, Cdc25Asv1 is modulated to achieve a therapeutic effect upon diseases in which regulation of Cdc25A is desirable. For example, in some embodiments, cancer may be treated by modulating Cdc25Asv1 to inhibit genes involved in oncogenesis. In other embodiments, disorders resulting from cellular response to exposure to ionizing radiation or ultra-violet light may be treated by modulating Cdc25Asv1 to either inhibit or induce proteins involved in response to DNA damage.

In some embodiments, the Cdc25Asv1 can be used to screen for Chk1 inhibitors. It has been shown that exposure of proliferating human cells to ultraviolet light results in a rapid decline in phosphatase activity of Cdc25A, accompanied by degradation of the Cdc25A protein (Mailand et al., Science 288:1425-1429, 2000). Further, it has been shown through use of a small interfering RNA specific to Chk1, that the degradation of Cdc25A is mediated by Chk1 (Xiao et al., J. Biol. Chem. 278:21767-21773, 2003). The Cdc25Asv1 protein can be used to assay for Chk1 inhibitors in an assay similar to the one described using Cdc25A in Xiao et al. For example, the effect of candidate Chk1 inhibitors can be assessed by measuring the amount of Cdc25Asv1 degradation that occurs in response to DNA damage. Cdc25Asv1 protein degradation can be measured, for example, by western blot or through use of electrochemiluminescence technology.

Cdc25Asv1 Nucleic Acids

Cdc25Asv1 nucleic acids contain regions that encode polypeptides comprising, consisting, or consisting essentially of SEQ ID NO:3. The Cdc25Asv1 nucleic acids have a variety of uses, such as use as a hybridization probe or PCR primer to identify the presence of Cdc25Asv1 nucleic acids; use as a hybridization probe or PCR primer to identify nucleic acids encoding for proteins related to Cdc25Asv1; and/or use for recombinant expression of Cdc25Asv1 polypeptides. In particular, Cdc25Asv1 polynucleotides do not have the polynucleotide region that consists of exon 6 of the Cdc25A gene.

Regions in Cdc25Asv1 nucleic acid molecules that do not encode Cdc25Asv1 or are not found in SEQ ID NO:2, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding Cdc25Asv1 related proteins from different sources. Obtaining nucleic acids encoding Cdc25Asv1 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook et al., in Molecular Cloning, A Laboratory Manual, 2d ed., Cold Spring Harbor Laboratory Press, 1989. PCR cloning techniques are described, for example, in White, Methods in Molecular Cloning, Vol. 67, Humana Press, 1997.

Cdc25Asv1 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons.” The translation of a particular codon into a particular amino acid is well known in the art (see, e.g., Lewin GENES IV, p. 119, Oxford University Press, 1990). Amino acids are encoded for by codons as follows:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asn=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2d ed., Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2d ed., Cold Spring Harbor Laboratory Press, 1989, and U.S. Pat. No. 5,480,784.

Ccd25Asv1 Probes

Probes for Cdc25Asv1 contain a region that can specifically hybridize to Cdc25Asv1 target nucleic acids, under appropriate hybridization conditions. Such probes can distinguish Cdc25Asv1 nucleic acids from non-target nucleic acids, in particular Cdc25A polynucleotides containing exon 6. Probes for Cdc25Asv1 can also contain nucleic acid regions that are not complementary to Cdc25A nucleic acids.

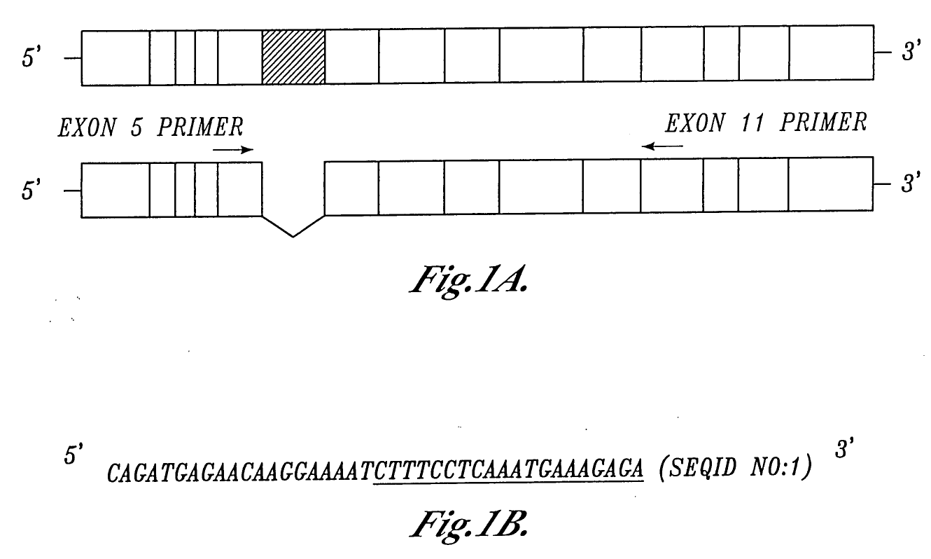

In embodiments where, for example, Cdc25Asv1 polynucleotide probes are used in hybridization assays to specifically detect the presence of Cdc25Asv1 polynucleotides in samples, the Cdc25Asv1 polynucleotides comprise at least 20 nucleotides of the Cdc25Asv1 sequence that corresponds to the novel exon junction polynucleotide region. In particular, for detection of Cdc25Asv1, the probe comprises at least 20 nucleotides of the Cdc25Asv1 sequence that corresponds to an exon junction polynucleotide created by the alternative splicing of exon 5 to exon 7 of the primary transcript of the Cdc25A gene (see FIGS. 1A and 1B and SEQ ID NO:1). For example, the polynucleotide sequence: 5° CAAGGAAAATCTTTCCTCAA 3′ [SEQ ID NO:4] represents one embodiment of such an inventive Cdc25Asv1 polynucleotide wherein a first 10 nucleotide region is complementary and hybridizable to the 3′ end of exon 5 of the Cdc25A gene and a second 10 nucleotide region is complementary and hybridizable to the 5′ end of exon 7 of the Cdc25A gene (see FIG. 1B).

In some embodiments, the first 20 nucleotides of a Cdc25Asv1 probe comprise a first continuous region of 5 to 15 nucleotides that is complementary and hybridizable to the 3′ end of exon 5 and a second continuous region of 5 to 15 nucleotides that is complementary and hybridizable to the 5′ end of exon 7.

In other embodiments, the Cdc25Asv1 polynucleotide comprises at least 40, 60, 80, or 100 nucleotides of the Cdc25Asv1 sequence that corresponds to a junction polynucleotide region created by the alternative splicing of exon 5 to exon 7 of the primary transcript of the Cdc25A gene. The Cdc25Asv1 polynucleotide is selected to comprise a first continuous region of at least 5 to 15 nucleotides that is complementary and hybridizable to the 3′ end of exon 5 and a second continuous region of at least 5 to 15 nucleotides that is complementary and hybridizable to the 5′ end of exon 7. As will be apparent to a person of skill in the art, a large number of different polynucleotide sequences from the region of exon 5 to exon 7 splice may be selected which will, under appropriate hybridization conditions, have the capacity to detectably hybridize to Cdc25Asv1 polynucleotides, and yet will hybridize to a much less extent, or not at all, to Cdc25A isoform polynucleotides wherein exon 5 is not spliced to exon 7.

Preferably, non-complementary nucleic acid that is present has a particular purpose such as being a reporter sequence or being a capture sequence. However, additional nucleic acid need not have a particular purpose as long as the additional nucleic acid does not prevent the Cdc25Asv1 nucleic acid from distinguishing between target polynucleotides, and non-target polynucleotides, including, but not limited to Cdc25A polynucleotides not comprising the exon 5 to exon 7 splice junction found in Cdc25Asv1.

Hybridization occurs through complementary nucleotide bases. Hybridization conditions determine whether two molecules, or regions, have sufficiently strong interactions with each other to form a stable hybrid. The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook et al., in Molecular Cloning, A Laboratory Manual, 2d ed., Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook et al., in Molecular Cloning, A Laboratory Manual, 2d ed., Cold Spring Harbor Laboratory Press, 1989. An example of high stringency conditions is as follows: prehybridization of filters containing DNA is carried out for 2 hours to overnight at 65° C. in buffer composed of 6× SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2× SSC, 0.1% SDS. This is followed by a wash in 0.1× SSC, 0.1% SDS at 50° C. for 45 minutes before autoradiography. Other procedures using conditions of high stringency would include, for example, either a hybridization step carried out in 5× SSC, 5× Denhardt's solution, 50% formamide at 42° C. for 12 to 48 hours or a washing step carried out in 0.2× SSPE, 0.2% SDS at 65° C. for 30 to 60 minutes.

CDC25ASV1 Recombiant Expression

Cdc25Asv1 polynucleotides, such as those comprising SEQ ID NO:2, can be used to make Cdc25Asv1 polypeptides. In particular, Cdc25Asv1 polypeptides can be expressed from recombinant nucleic acids in a suitable host or in vitro using a translation system. Recombinantly expressed Cdc25Asv1 polypeptides can be used, for example, in assays to screen for compounds that bind Cdc25Asv1. Alternatively, Cdc25Asv1 polypeptides can also be used to screen for compounds that bind to one or more Cdc25A isoforms, but do not bind to Cdc25Asv1.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXTl (Stratagene), pSG5 (Stratagene), pCMVLac1 (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460). Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pPICZ (Invitrogen), pYES2 (Invitrogen), and Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26) MRC-5 (ATCC CCL 171), and HEK 293 cells (ATCC CRL-1573).

To enhance expression in a particular host, it may be useful to modify the sequence provided in SEQ ID NO:2 to take into account codon usage of the host. Codon usages of different organisms are well known in the art (see, Ausubel, Current Protocols in Molecular Biology, John Wiley, 1987-1998, Supplement 33, Appendix 1C).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

Cdc25Asv1 Polypeptides

Cdc25Asv1 polypeptides contain an amino acid sequence comprising, consisting, or consisting essentially of SEQ ID NO:3. Cdc25Asv1 polypeptides have a variety of uses, such as providing a marker for the presence of Cdc25Asv1; use as an immunogen to produce antibodies binding to Cdc25Asv1; use as a target to identify compounds binding selectively to Cdc25Asv1; or use in an assay to identify compounds that bind to one or more isoforms of Cdc25A but do not bind to or interact with Cdc25Asv1.

In chimeric polypeptides containing one or more regions from Cdc25Asv1 and one or more regions not from Cdc25Asv1, the region(s) not fromCdc25Asv1 can be used, for example, to achieve a particular purpose or to produce a polypeptide that can substitute for Cdc25Asv1, or fragments thereof. Particular purposes that can be achieved using chimeric Cdc25Asv1 polypeptides include providing a marker for Cdc25Asv1 or activity, modulating the activity of Cdc2, and modulating the G1/S and/or G2/M cell cycle transition. Chimeric Cdc25Asv1 polypeptides can also be used as a substrate for screening potential therapeutics that target Chk1 activity.

Polypeptides can be produced using standard techniques including those involving chemical synthesis and those involving biochemical synthesis. Techniques for chemical synthesis of polypeptides are well known in the art (see, e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook et al., in Molecular Cloning, A Laboratory Manual, 2d ed., Cold Spring Harbor Laboratory Press, 1989.

Functional Cdc25Asv1

Functional Cdc25Asv1 is a protein isoform of Cdc25A. The identification of the amino acid and nucleic acid sequences of Cdc25Asv1 provide tools for obtaining functional proteins related to Cdc25Asv1 from other sources, for producing Cdc25Asv1 chimeric proteins, and for producing functional derivatives of SEQ ID NO:3.

Cdc25Asv1 polypeptides can be readily identified and obtained based on their sequence similarity to Cdc25Asv1 (SEQ ID NO:3). In particular, Cdc25Asv1 lacks a 119 base pair region corresponding to exon 6 of the Cdc25A. The deletion of exon 6 does not disrupt the protein reading frame as compared to the Cdc25A reference sequence (NM_{—}001789.1). Therefore, Cdc25Asv1 polypeptide lacks an internal 40 amino acid region corresponding to the amino acid region encoded by exon 6 as compared to the Cdc25A reference sequence (NM_{—}001789.1).

Both the amino acid and nucleic acid sequences of Cdc25Asv1 can be used to help identify and obtain Cdc25Asv1 polypeptides. For example, SEQ ID NO:2 can be used to produce degenerative nucleic acid probes or primers for identifying and cloning nucleic acid polynucleotides encoding for a Cdc25Asv1 polypeptide. In addition, polynucleotides comprising, consisting, or consisting essentially of SEQ ID NO:2 or fragments thereof, can be used under conditions of moderate stringency to identify and clone nucleic acids encoding Cdc25Asv1 polypeptides from a variety of different organisms.

The use of degenerative probes and moderate stringency conditions for cloning is well known in the art. Examples of such techniques are described by Ausubel, Current Protocols in Molecular Biology, John Wiley, 1987-1998, and Sambrook et al., in Molecular Cloning, A Laboratory Manual, 2d ed., Cold Spring Harbor Laboratory Press, 1989.

Starting with Cdc25Asv1 obtained from a particular source, derivatives can be produced. Such derivatives include polypeptides with amino acid substitutions, additions and deletions. Changes to Cdc25Asv1 to produce a derivative having essentially the same properties should be made in a manner not altering the tertiary structure of Cdc25Asv1.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (see, Ausubel, Current Protocols in Molecular Biology, John Wiley, 1987-1998, Supplement 33 Appendix 1C).

Cdc25Asv1 Antibodies

Antibodies recognizing Cdc25Asv1 can be produced using a polypeptide containing SEQ ID NO:3, or a fragment thereof, as an immunogen. Preferably, a Cdc25Asv1 olypeptide used as an immunogen consists of a polypeptide of SEQ ID NO:3 fragment having at least 10 contiguous amino acids in length corresponding to the polynucleotide region representing the junction resulting from the splicing of exon 5 to exon 7 of the Cdc25A gene.

In some embodiments where, for example, Cdc25Asv1 polypeptides are used to develop antibodies that bind specifically to Cdc25Asv1 and not to other isoforms of Cdc25A, the Cdc25Asv1 polypeptides comprise at least 10 amino acids of the Cdc25Asv1 polypeptide sequence corresponding to a junction polynucleotide region created by the alternative splicing of exon 5 to exon 7 of the primary transcript of the Cdc25A gene (see FIG. 1B). For example, the amino acid sequence: amino terminus- “ENKENLSSNE”-carboxy terminus [SEQ ID NO:5] represents one embodiment of such an inventive Cdc25Asv1 polypeptide wherein a first 5 amino acid region is encoded by nucleotide sequence at the 3′ end of exon 5 of the Cdc25A gene and a second 5 amino acid region is encoded by the nucleotide sequence directly after the novel splice junction. Preferably, at least 10 amino acids of the Cdc25Asv1 polypeptide comprise a first continuous region of 2 to 8 amino acids that is encoded by nucleotides at the 3′ end of exon 5 and a second continuous region of 2 to 8 amino acids that is encoded by nucleotides at the 5′ end of exon 7.

In other embodiments, Cdc25Asv1 specific antibodies are made using an Cdc25Asv1 polypeptide that comprises at least 20, 30, 40, or 50 amino acids of the Cdc25Asv1 sequence that corresponds to a junction polynucleotide region created by the alternative splicing of exon 5 to exon 7 of the primary transcript of the Cdc25A gene. In each case, the Cdc25Asv1 polypeptides are selected to comprise a first continuous region of at least 5 to 15 amino acids that is encoded by nucleotides at the 3′ end of exon 5 and a second continuous region of 5 to 15 amino acids that is encoded by nucleotides directly after the novel splice junction.

Antibodies to Cdc25Asv1 have different uses, such as, for example, to identify the presence of Cdc25Asv1, to isolate Cdc25Asv1 polypeptides, and to determine the effectiveness of Cdc25Asv1 ligands. Identifying the presence of Cdc25Asv1 can be used, for example, to identify cells producing Cdc25Asv1. Such identification provides an additional source of Cdc25Asv1 and can be used to distinguish cells known to produce Cdc25Asv1 from cells that do not produce Cdc25Asv1. For example, antibodies to Cdc25Asv1 can distinguish human cells expressing Cdc25Asv1 from human cells not expressing Cdc25Asv1 or non-human cells (including bacteria) that do not express Cdc25Asv1. Such Cdc25Asv1 antibodies can also be used to determine the effectiveness of Cdc25Asv1 ligands, using techniques well known in the art, to detect and quantify changes in the protein levels of Cdc25Asv1 in cellular extracts, and in situ immunostaining of cells and tissues.

Techniques for producing and using antibodies are well known in the art. Examples of such techniques are described in Ausubel, Current Protocols in Molecular Biology, John Wiley, 1987-1998; Harlow et al., Antibodies, A Laboratory Manual, Cold Spring Harbor Laboratory, 1988; and Kohler et al., Nature 256:495-7, 1975.

Cdc25Asv1 Binding Assay

Cdc25Asv1, or fragments thereof, can be used in binding studies to identify agents or compounds useful for modulating (inhibiting or enhancing) the cell cycle. A number of compounds that modulate Cdc25A activity may be identified in such binding studies, such as inhibitors of the catalytic activity of tyrosine specific phosphatases, blocking agents which interfere with the interaction or binding of the tyrosine specific phosphatase with Cyclin B or the CyclinB/Cdc2 complex, or agents which interfere directly with the catalytic activity of the Cdc25A phosphatase. Methods for screening compounds for their effects on Cdc25A activity have been disclosed (see for example, U.S. Pat. No. 5,695,950). A person skilled in the art may use these methods to screen Cdc25Asv1 polypeptides for compounds that bind to, and in some cases functionally alter Cdc25Asv1.

In one embodiment, Cdc25Asv1, or a fragment thereof, can be used in binding studies with Cdc25A isoform protein, or a fragment thereof, to identify compounds that bind to and/or interact with Cdc25Asv1 and other Cdc25A isoforms, or alternatively, that bind to and/or interact with one or more other Cdc25A isoforms and not with Cdc25Asv1. Such binding studies can be performed using different formats, including competitive and non-competitive formats. Further competition studies can be carried out using additional compounds determined to bind to Cdc25Asv1, or other Cdc25A isoforms.

The particular Cdc25Asv1 sequence involved in ligand binding can be identified using labeled compounds that bind to the protein and different protein fragments. Different strategies can be employed to select fragments to be tested in order to identify the binding region. Examples of such strategies include testing consecutive fragments of about 15 amino acids or longer in length starting at the N-terminus. If longer length fragments are tested, a fragment binding to a compound can be subdivided to further locate the binding region. Fragments used for binding studies can be generated using recombinant nucleic acid techniques.

In some embodiments, binding studies are performed using Cdc25Asv1 expressed from a recombinant nucleic acid. Alternatively, recombinantly expressed Cdc25Asv1 consists of the SEQ ID NO:3 amino acid sequence.

Binding assays can be performed using individual compounds or preparations containing different numbers of compounds. A preparation containing different numbers of compounds having the ability to bind to Cdc25Asv1 can be divided into smaller groups of compounds that can be tested to identify the compound(s) binding to Cdc25Asv1 or Cdc25A, respectively.

Binding assays can be performed using recombinantly produced Cdc25Asv1 present in different environments. Such environments include, for example, cell extracts and purified cell extracts containing a Cdc25Asv1 recombinant nucleic acid; and also include, for example, the use of a purified Cdc25Asv1 polypeptide produced by recombinant means which is introduced into different environments.

In one embodiment of the invention, a binding method is provided for screening for a compound able to bind selectively to Cdc25Asv1. The method comprises the following steps: providing a Cdc25Asv1 polypeptide comprising SEQ ID NO:3; providing a Cdc25A isoform polypeptide that is not Cdc25Asv1; contacting the Cdc25Asv1 polypeptide and the Cdc25A1 isoform polypeptide that is not Cdc25Asv1 with a test preparation comprising one or more test compounds; and then determining the binding of the test preparation to the Cdc25Asv1 polypeptide and to the Cdc25A isoform polypeptide that is not Cdc25Asv1, wherein a test preparation that binds to the Cdc25Asv1 polypeptide, but does not bind to Cdc25A isoform polypeptide that is not Cdc25Asv1, contains one or more compounds that selectively bind to Cdc25Asv1.

In another embodiment of the invention, a binding method is provided for screening for a compound able to bind selectively to a Cdc25A isoform polypeptide that is not Cdc25Asv1. The method comprises the following steps: providing a Cdc25Asv1 polypeptide comprising SEQ ID NO:3; providing a Cdc25A isoform polypeptide that is not Cdc25Asv1; contacting the Cdc25Asv1 polypeptide and the Cdc25A isoform polypeptide that is not Cdc25Asv1 with a test preparation comprising one or more test compounds; and then determining the binding of the test preparation to the Cdc25Asv1 polypeptide and the Cdc25A isoform polypeptide that is not Cdc25Asv1, wherein a test preparation that binds the Cdc25A isoform polypeptide that is not Cdc25Asv1, but does not bind Cdc25Asv1, contains a compound that selectively binds the Cdc25A isoform polypeptide that is not Cdc25Asv1.

The above-described selective binding assays can also be performed with a polypeptide fragment of Cdc25Asv1, wherein the polypeptide fragment comprises at least 10 consecutive amino acids that are encoded by a nucleotide sequence that bridges the junction created by the splicing of the 3′ end of exon 5 to the 5′ end of exon 7 of Cdc25A. Similarly, the selective binding assays may also be performed using a polypeptide fragment of a Cdc25A isoform polypeptide that is not Cdc25Asv1, wherein the polypeptide fragment comprises at least 10 consecutive amino acids that are coded by: a) a nucleotide sequence that is contained within exon 6 of the Cdc25A gene; or b) a nucleotide sequence that bridges the junction created by the splicing of the 3′ end of exon 5 to the 5′ end of exon 7.

Cdc25A Functional Assays

Cdc25A encodes a tyrosine phosphatase that triggers progression from G1 to S phase of the eukaryotic cell cycle by binding to, and directly dephosphorylating Cdc2 (Gauteir et al., Cell 67:197-211, 1991). The phosphatase activity of Cdc25A depends on phosphorylation of the Cdc25A polypeptide by the Chk1 kinase (Xiao et al., J. Biol. Chem. 278:21767-21773, 2003). It has also been shown that Cdc25A is inhibited by pl3-Sucl (U.S. Pat. No. 5,441,880). In view of the foregoing, the identification of Cdc25Asv1 as a splice variant of Cdc25A provides a means for screening for compounds that bind to Cdc25Asv1 protein thereby altering one or more functions of the Cdc25Asv1 polypeptide. For example, a compound that binds to Cdc25Asv1 may inhibit the ability of the Cdc25Asv1 polypeptide to trigger progression from G1 to S phase. Assays involving a functional Cdc25Asv1 polypeptide can be employed for different purposes, such as selecting for compounds that modulate Cdc25Asv1 phosphatase activity; evaluating the ability of a compound to effect the phosphorylation of Cdc25Asv1; and mapping the activity of the Cdc25Asv1 region.

Cdc25Asv1 activity can be measured using different techniques such as: measuring the ability of Cdc25Asv1 to trigger cell cycle progression, measuring the tyrosine phosphatase activity of Cdc25Asv1, detecting a change in the intracellular conformation of Cdc25Asv1; detecting a change in the intracellular location of Cdc25Asv1; detecting the amount of binding of Cdc25Asv1 to Cyclin B and Cdc2, and detecting the effect of inhibitors such as pl3-Sucl on Cdc25Asv1, as compared to Cdc25A. In addition, the Cdc25Asv1 polypeptide can be assayed for its effect on modulating DNA synthesis after exposure to genotoxic agents (such as, for example, ionizing radiation).

With respect to phosphatase activity, tyrosine phosphatase assays may be used to determine whether Cdc25Asv1 can function as a phosphatase with respect to various substrates such as Cdc2 and a Cdc2/Cyclin B complex (see for example, U.S. Pat. No. 5,441,880). For example, phosphorylation levels of various substrates, such as Cdc2, can be assessed by gel-mobility shift assays, or by loss of cross-reactivity with an antibody directed against a Cdc2 peptide comprising the tyrosine-15 residue (as described in U.S. Pat. No. 5,294,538). The ability of various inhibitors, such as, for example, pl3-Suc1 on Cdc25Asv1 phosphatase activity can also be assessed in the above assay. Cdc25Asv1 function can also be assessed by its ability to activate Cdc2 kinase activity as measured in a Histone Hi kinase assay (described in U.S. Pat. No. 5,441,880).

Recombinantly expressed Cdc25Asv1 can be used to facilitate the determination of whether a compound binds to and/or modulates Cdc25Asv1. For example, Cdc25Asv1 can be expressed by an expression vector in a cell line and used in a co-culture growth assay, such as described in WO 99/59037, to identify compounds that bind to Cdc25Asv1. By way of another illustrative example, Cdc25Asv1 can be expressed by an expression vector in a human kidney cell line 293 and used in a co-culture growth assay, such as described in U.S. patent application Ser. No. 20020061860, to identify compounds that bind to Cdc25Asv1.

Cdc25Asv1 functional assays can be performed using recombinantly produced Cdc25Asv1 present in different environments. Such environments include, for example, cell extracts and purified cell extracts containing Cdc25Asv1 expressed from recombinant nucleic acid; and the use of purified Cdc25Asv1 produced by recombinant means that is introduced into a different environment suitable for measuring binding or phosphatase activity.

Cdc25A functional assays can be also be performed using cells that over-produce Cdc25Asv1. A preparation containing different compounds where one or more compounds affect Cdc25Asv1 in cells over-producing Cdc25Asv1 as compared to control cells containing an expression vector lacking Cdc25Asv1 coding sequences, can be divided into smaller groups of compounds to identify the compound(s) affecting Cdc25Asv1.

Modulating Ccd25Asv1 Expression

Cdc25Asv1 expression may be modulated as a means for increasing or decreasing Cdc25Asv1 activity. Such modulation includes inhibiting the activity of nucleic acids encoding the Cdc25Asv1 target to reduce Cdc25Asv1 protein or polypeptide expression, or supplying Cdc25A nucleic acids to increase the level of expression of the Cdc25A target polypeptide thereby increasing Cdc25A activity.

Inhibition of Cdc25Asv1 Activity

Cdc25Asv1 nucleic acid activity may be inhibited using nucleic acids recognizing Cdc25Asv1 nucleic acid and affecting the ability of such nucleic acid to be transcribed or translated. Inhibition of Cdc25Asv1 nucleic acid activity may be used, for example, in target validation studies.

A preferred target for inhibiting Cdc25Asv1 is mRNA stability and translation. The ability of Cdc25Asv1 mRNA to be translated into a protein may be effected by compounds such as anti-sense nucleic acid, RNA interference (RNAi) and enzymatic nucleic acid.

Anti-sense nucleic acid is capable of hybridizing to a region of a target mRNA. Depending on the structure of the anti-sense nucleic acid, anti-sense activity may be brought about by different mechanisms such as blocking the initiation of translation, preventing processing of mRNA, hybrid arrest, and degradation of mRNA by RNAse H activity.

RNA inhibition (RNAi) using shRNA or siRNA molecules may also be used to prevent protein expression of a target transcript. This method is based on the interfering properties of double-stranded RNA derived from the coding regions of the gene that disrupt the synthesis of protein from transcribed RNA.

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Modified and unmodified nucleic acids can be used as anti-sense molecules, RNAi and ribozymes. Different types of modifications can affect certain anti-sense activities such as the ability to be cleaved by RNAse H, and can alter nucleic acid stability. For example, a system to probe for suitable sites in mRNA for antisense oligonucleotide targeting has been established using Rnase H cleavage as an indicator for accessibility of sequences within transcripts (see, Scherr et al., NAR 26:5079-5085, 1998). The system described by Scherr et al. involves adding a mixture of oligonucleotides that are complementary to certain regions of a transcript, such as a Cdc25Asv1 transcript, to cell extracts and exposing the sample to Rnase H. RT-PCR is then used to show which oligos actually had access to the transcript and hybridized in order to create an Rnase H vulnerable site. This technique can be combined with computer assisted sequence selection. Illustrative examples of the successful use of different anti-sense molecules and ribozymes are provided in U.S. Pat. Nos. 5,849,902; 5,859,221; 5,852,188; and 5,616,459. Examples of organisms in which RNAi has been used to jnhibit expression of a target gene include: C. elegans (Tabara, et al., Cell 99:123-32, 1999; Fire, et al., Nature 391:806-11, 1998), plants (Hamilton and Baulcombe, Science 286:950-52, 1999), Drosophila (Hammond et al., Science 293:1146-50, 2001; Misquitta and Patterson, Proc. Nat. Acad. Sci. 96:1451-56, 1999; Kennerdell and Carthew, Cell 95:1017-26, 1998), and mammalian cells (Bernstein et al., Nature 409:363-6, 2001; Elbashir et al., Nature 411:494-8, 2001).

Increasing Cdc25Asv1 Expression

Nucleic acids encoding Cdc25Asv1 can be used, for example, to cause an increase in Cdc25A activity or to create a test system (e.g., a transgenic animal) for screening for compounds affecting Cdc25Asv1 expression. Nucleic acids can be introduced and expressed in cells present in different environments.

Guidelines for pharmaceutical administration in general are provided in, for example, Remington's Pharmaceutical Sciences, 18^{th} ed., supra, and Modern Pharmaceutics, 2d ed., supra. Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

Examples are provided below to further illustrate different features and advantages of the present invention. The examples also illustrate useful methodology for practicing the invention. These examples do not limit the claimed invention.

### EXAMPLE 1

### Identification Of Cdc25Asv1 Using Microarrays

To identify variants of the “normal” splicing of exon regions encoding Cdc25A, an exon junction microarray, comprising probes complementary to each splice junction resulting from splicing of the 15 exon coding sequences in Cdc25A heteronuclear RNA (hnRNA), was hybridized to a mixture of labeled nucleic acid samples prepared from 44 different human tissue and cell line samples. Exon junction microarrays and methods of analysis are described by Johnson et al. (Science 302:2141-44 (2003), including Supporting Online Materials) and in International Patent Application Nos. WO 02/18646 and WO 02/16650. Materials and methods for preparing hybridization samples from purified RNA, hybridizing a microarray, detecting hybridization signals, and data analysis are described in van't Veer et al. (Nature415:530-536, 2002) and Hughes etal. (Nature Biotechnol. 19:342-7, 2001). Inspection of the exon junction microarray hybridization data (not shown) suggested that the structure of one of the exon junctions of Cdc25A mRNA was altered in some of the tissues examined, indicating the possible presence of Cdc25A splice variant mRNA populations. Reverse transcription and polymerase chain reactions (RT-PCR) were then performed using oligonucleotide primers complementary to exons 5 and 11 to confirm the exon junction array results and to allow the sequence structure of the splice variant to be determined.

### EXAMPLE 2

### Confirmation Of Cdc25Asv1 Using RT-PCR

The structure of Cdc25A mRNA in the region corresponding to exons 5 to 11 was determined for a panel of human tissue and cell line samples using an RT-PCR based assay. PolyA purified mRNA isolated from 44 different human tissue and cell line samples was obtained from BD Biosciences Clontech (Palo Alto, Calif.), Biochain Institute, Inc. (Hayward, Calif.), and Ambion Inc. (Austin, Tex.). RT-PCR primers were selected that were complementary to sequences in exon 5 and exon 11 of the reference exon coding sequences in Cdc25A (NM_{—}001789.1). Based upon the nucleotide sequence of Cdc25A mRNA, the Cdc25A exon 5 and exon 11 primer set (hereafter Cdc25A₅₋₁₁ primer set) was expected to amplify a 668 base pair amplicon representing the “reference” Cdc25A mRNA region. The Cdc25A exon 5 forward primer has the sequence:

| | |
|---|---|
| 5′ CTCATCGACCCAGATGAGAACAAGGAAA 3′. | [SEQ ID NO:6] |

The Cdc25A exon 11 reverse primer has the sequence:

| | |
|---|---|
| 5′ TCCTGATGTTTCCCAGCAACTGTATGAA 3′. | [SEQ ID NO:7] |

25 ng of polyA mRNA from each tissue was subjected to a one-step reverse transcription-PCR amplification protocol using the Qiagen, Inc. (Valencia, Calif.), One-Step RT-PCR kit, using the following cycling conditions:
50° C. for 30 minutes;
95° C. for 15 minutes;
35 cycles of:
   94° C. for 30 seconds;
   63.5° C. for 40 seconds;
   72° C. for 50 seconds; then
   72° C. for 10 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were sequenced from each end (using the same primers used for RT-PCR) by Qiagen Genomics, Inc. (Bothell, Wash.).

At least two different RT-PCR amplicons were obtained from human mRNA samples using the Cdc25A₅₋₁₁ primer set (data not shown). Of the 44 different human tissues and cell line samples assayed, every sample that exhibited the expected amplicon size of 668 base pairs for normally spliced Cdc25A also exhibited an amplicon of about 489 base pairs.

The tissues assayed in which Cdc25A mRNA was detected are listed in Table 1:

TABLE 1

| Sample | Cdc25A | Cdc25Asv1 |
|---|---|---|
| Heart | − | − |
| Kidney | − | − |
| Liver | − | − |
| Brain | − | − |
| Placenta | − | − |
| Lung | − | − |
| Brain-fetal | − | − |
| Leukemia Promyelocytic (HL-60) | − | − |
| Adrenal Medulla | − | − |
| Fetal Liver | − | − |
| Salivary Gland | − | − |
| Lymphoma-Burkitts (Raji) | + | + |
| Spinal Cord | − | − |
| Lymph Node | − | − |
| Fetal Kidney | + | + |
| Uterus | − | − |
| Spleen | − | − |
| Brain-thalamus | + | + |
| Fetal Lung | + | + |
| Testis | + | + |
| Melanoma (G361) | + | + |
| Lung carcinoma (A549) | + | + |
| Pancreas | − | − |
| Skeletal Muscle | − | − |
| Brain-cerebellum | − | − |
| Stomach | − | − |
| Trachea | − | − |
| Thyroid | − | − |
| Bone Marrow | + | + |
| Brain-amygdala | − | − |
| Brain-caudate nucleus | − | − |
| Brain-corpus callosum | − | − |
| Ileocecum | − | − |
| Adrenal medulla | − | − |
| Brain-cerebral cortex | + | + |
| Colon-descending | + | + |
| Prostate | + | + |
| Duodenum | + | + |
| Epididymis | − | − |
| Brain-hippocampus | − | − |
| Ileum | + | + |
| Heart-interventricular septum | − | − |
| Jejunum | − | − |
| Rectum | − | − |

As shown in Table 1, samples exhibiting both the 668 base pair and the 489 base pair amplicons included Burketts Lymphoma, fetal kidney, brain (thalamus), fetal lung, testis, melanoma (G361), lung carcinoma (A549), bone marrow, brain (cerebral cortex), descending colon, prostate, duodenum and ileum. Tissues and cell lines that did not exhibit expression of Cdc25A included heart, kidney, liver, brain, placenta, lung, fetal brain, promyelocytic leukemia (HL-60), adrenal medulla, fetal liver, salivary gland, spinal cord, lymph node, uterus, spleen, pancreas, skeletal muscle, brain (cerebellum), stomach, trachea, thyroid, brain (amygdala, caudate nucleus, corpus callosum, hippocampus), ileocecum, epididymis, heart (interventricular septum), jejunum, and rectum.

Sequence analysis of the 668 base pair amplicon amplified using the Cdc25A exon 5-11 primer set revealed that this amplicon form results from the splicing of exon 5 of the Cdc25A mRNA to exon 7; that is, exon 6 coding sequence is completely absent. Thus, the RT-PCR results confirmed the junction probe microarray data reported in Example 1, suggesting that Cdc25A mRNA is composed of a mixed population of molecules wherein at least one of the Cdc25A mRNA splice junctions are altered.

### EXAMPLE 3

### Cloning Of Cdc25Asv1

Microarray and RT-PCR data indicate that in addition to the normal Cdc25A reference mRNA sequence, NM_{—}001789.1, encoding Cdc25A protein, NP_{—}001780.1, a novel splice variant form of Cdc25A mRNA also exists in many tissues.

Clones having a nucleotide sequence comprising the splice variant identified in Example 2 (hereafter referred to as Cdc25Asv1) are isolated using a 5′ “forward” Cdc25A primer and a 3′ “reverse” Cdc25A primer, to amplify and clone the entire Cdc25Asv1 mRNA coding sequence. The 5′ “forward” primer is designed for isolation of full length clones corresponding to the Cdc25Asv1 splice variant and has the nucleotide sequence of 5′ATGGAACTGGGCCCGAGCCCCGCACCGC 3′ [SEQ ID NO:8]. The 3′ “reverse” primer is designed for isolation of full length clones corresponding to the Cdc25Asv1 splice variant and has the nucleotide sequence of 5′ TCAGAGCTTCTTCAGACGACTGTACATC 3′ [SEQ ID NO:9].

RT-PCR

The Cdc25Asv1 cDNA sequence is cloned using a combination of reverse transcription (RT) and polymerase chain reaction (PCR). More specifically, about 25 ng of testis polyA mRNA (BD Biosciences Clontech, Palo alto, Calif.) is reverse transcribed using Superscript II (Gibco/Invitrogen, Carlsbad, CA) and oligo d(T) primer (RESGEN/Invitrogen, Huntsville, Al.) according to the Superscript II manufacturer's instructions. For PCR, 1 μl of the completed RT reaction is added to 40 μl of water, 5 μl of 10× buffer, 1 μl of dNTPs and 1 μl of enzyme from the Clontech (Palo Alto, Calif.) Advantage 2 PCR kit. PCR is done in a Gene Amp PCR System 9700 (Applied Biosystems, Foster City, Calif.) using the Cdc25A “forward” [SEQ ID NO:8] and “reverse” [SEQ ID NO:9] primers. After an initial 94° C. denaturation of 1 minute, 35 cycles of amplification are performed using a 30 second denaturation at 94° C. followed by a 40 second annealing at 63.5° C. and a 50 second synthesis at 72° C. The 35 cycles of PCR are followed by a 10-minute extension at 72° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determine if the PCR has yielded products of 1452 base pairs, the expected size for Cdc25Asv1 mRNA. The remainder of the 50 μl PCR reaction from testis is purified using the QIAquik Gel extraction Kit (Qiagen, Valencia, Calif.) following the QIAquik PCR Purification Protocol provided with the kit. About 50 μl of product obtained from the purification protocol is concentrated to about 6 μl by drying in a Speed Vac Plus (SC110A, from Savant, Holbrook, N.Y.) attached to a Universal Vacuum System 400 (also from Savant) for about 30 minutes on medium heat.

Cloning Of RT-PCR Products

About 4 μl of the 6 μl of purified Cdc25Asv1 RT-PCR product from testis is used in a cloning reaction using the reagents and instructions provided with the TOPO TA cloning kit (Invitrogen, Carlsbad, Calif.). About 2 μl of the cloning reaction is used following the manufacturer's instructions to transform TOP10 chemically competent E. coli provided with the cloning kit. After the 1 hour recovery of the cells in SOC medium (provided with the TOPO TA cloning kit), 200 μl of the mixture is plated on LB medium plates (Sambrook, et al., in Molecular Cloning, A Laboratory Manual, 2d ed., Cold Spring Harbor Laboratory Press, 1989) containing 100 μg/ml Ampicillin (Sigma, St. Louis, Mo.) and 80 μg/ml X-GAL (5-Bromo-4-chloro-3-indoyl B-D-galactoside, Sigma, St. Louis, Mo.). Plates are incubated overnight at 37° C. White colonies are picked from the plates into 2 ml of 2× LB medium. These liquid cultures are incubated overnight on a roller at 37° C. Plasmid DNA is extracted from these cultures using the Qiagen (Valencia, Calif.) Qiaquik Spin Miniprep kit. Twelve putative Cdc25Asv1 clones are identified and prepared for a PCR reaction to confirm the presence of the expected Cdc25Asv1 exon 5 to exon 7 splice variant structure. A 25 μl PCR reaction is performed as described above (RT-PCR section) to detect the presence of Cdc25Asv1, except that the reaction includes miniprep DNA from the TOPO TA/Cdc25Asv1 ligation as a template. About 10 μl of the 25 μl PCR reaction is run on a 1% Agarose gel and the DNA bands generated by the PCR reaction are visualized and photographed on a UV light box to determine which minipreps samples have PCR product of the size predicted for the corresponding Cdc25Asv1 splice variant mRNA. Clones having the Cdc25Asv1 structure are identified based upon amplification of an amplicon band of 1452 base pairs, whereas a normal reference Cdc25A clone will give rise to an amplicon band of 1571 base pairs. DNA sequence analysis of the Cdc25Asv1 cloned DNA confirms a polynucleotide sequence representing an in-frame deletion of exon 6 which is a variant of the reference Cdc25A DNA.

The polynucleotide sequence of Cdc25A mRNA (SEQ ID NO:2) contains an open reading frame that encodes a Cdc25Asv1 protein (SEQ ID NO:3) similar to the reference Cdc25A protein (NP_{—}001780.1), but lacking amino acids encoded by a 119 base pair region corresponding to exon 6 of the full length coding sequence of the reference Cdc25A mRNA (NM_{—}001789.1). The deletion of the 119base pair region does not change the protein translation reading frame in comparison to the reference Cdc25A protein reading frame. Therefore, the Cdc25Asv1 protein is missing an internal 40 amino acid region as compared to the reference Cdc25A (NP_{—}001780.1).

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

1. A purified human nucleic acid molecule comprising SEQ ID NO:2, or the complement thereof.

2. The purified nucleic acid molecule of claim 1, wherein said nucleic acid molecule comprises a region encoding SEQ ID NO:3.

3. The purified nucleic acid molecule of claim 1, wherein said nucleotide sequence encodes a polypeptide consisting of SEQ ID NO:3.

4. A purified polypeptide comprising SEQ ID NO:3.

5. The polypeptide of claim 4, wherein said polypeptide consists of SEQ ID NO:3.
